(19) United States
(12) Patent Application Publication
(10) Pub. No.: US 2026/0325415 A1
(43) Pub. Date: Oct. 22, 2026

(54) COSMETIC FORMULATIONS AND USES THEREOF

(71) Applicant: Coty Inc., New York, NY (US)

(72) Inventors: Alice Burzava, MONACO (MC); Karine Barbe, MONACO (MC); Muriel Pujos, Nice (FR); Karine Julien, St André de la Roche (FR)

(21) Appl. No.: 19/161,886

(22) PCT Filed: Mar. 1, 2024
(86) PCT No.: PCT/US2024/018170

§ 371 Date: Sep. 3, 2025

(30) Foreign Application Priority Data
Mar. 3, 2023 (FR) 2301986

(51) Int. Cl. A61K 8/06; A61K 8/37; A61K 8/44; A61K 8/49; A61K 8/73; A61K 8/81; A61K 8/99; A61Q 19/00

(52) U.S. Cl. CPC A61K 8/062; A61K 8/375; A61K 8/442; A61K 8/4973; A61K 8/73; A61K 8/8188; A61K 8/99; A61Q 19/00; A61K 2800/10

(57) ABSTRACT

Various aspects disclosed relate to a cosmetic formula. The cosmetic formula includes a gel mixture in a range of from about 0.1 wt % to about 2 wt % of the cosmetic formula. The gel mixture includes sphingomas ferment extract; xanthan gum; and a mixture of hydroxyethyl aery late/sodium acryloyldimethyl taurate copolymer & sorbitan isostearate. The cosmetic formula also includes a surfactant in a range of from about 0.1 wt % to about 2 wt % of the cosmetic formula and comprising sodium stearoyl glutamate. The cosmetic formula also includes an oil phase in a range of from about 2 wt % to about 15 wt % of the cosmetic formula. The oil phase includes caprylic capric triglyceride; a C15-19 alkane. The formulation further includes an active component. A pH of the cosmetic formula is in a range of from about 5 to about 7. A viscosity of the cosmetic formula is in a range of from about 1500 cPs to about 5000 cPs. The cosmetic formula is sufficiently stable for at least 2 months at a temperature of about 45 °C.

### BACKGROUND

Cosmetic formulations can be used to deliver any variety of beneficial effects to a user. Often the beneficial effect is provided by an active component. The ability to increase the diversity and amount of active component can increase the utility of the cosmetic formulation.

### SUMMARY OF THE INVENTION

Various aspects disclosed relate to a cosmetic formula. The cosmetic formula includes a gel mixture in a range of from about 0.1 wt % to about 2 wt % of the cosmetic formula. The gel mixture includes sphingomas ferment extract; xanthan gum, and a mixture of hydroxyethyl acrylate/sodium acryloyldimethyl taurate copolymer & sorbitan isostearate. The cosmetic formula also includes a surfactant in a range of from about 0.1 wt % to about 2 wt % of the cosmetic formula and comprising sodium stearoyl glutamate. The cosmetic formula also includes an oil phase in a range of from about 2 wt % to about 15 wt % of the cosmetic formula. The oil phase includes caprylic capric triglyceride; a C15-19 alkane. The cosmetic formula further includes an active component. A pH of the cosmetic formula is in a range of from about 5 to about 7. A viscosity of the cosmetic formula is in a range of from about 1500 cPs to about 5000 cPs. The cosmetic formula is sufficiently stable for at least 2 months at a temperature of about 45 °C.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Disclosed herein a omnipotent cosmetic formula. The cosmetic formula can be omnipotent in that it is capable of having one or more beneficial effects on a user's skin. The omnipotent cosmetic formula can take the form of a serum. Examples of uses for the cosmetic formula include use for skin fine lines reduction, improving skin firmness, evening skin tone, strengthening skin barrier, hydrating skin, enhancing skin natural radiance, or a combination thereof.

The properties of the cosmetic formula are a function of its components. Specifically, the ability to locate an active component in an oil phase of the formula.

The gel mixture can give the cosmetic formula sufficient properties relating to viscosity, feel/texture, and the like. The texture of the formulation is similar to a serum and can have a substantially translucent appearance. The gel mixture as a whole is in a range off from about 0.1 wt % to about 2 wt % of the cosmetic formula, about 0.3 wt % to about 1 wt %, less than, equal to, or greater than about 0.1 wt %, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or about 2 wt %. The gel mixture includes sphingomas ferment extract, xanthan gum; and a mixture of hydroxyethyl acrylate/sodium acryloyldimethyl taurate copolymer & sorbitan isostearate. Within the cosmetic formula, sphingomas ferment extract is in a range of from about 0.05 wt % to about 0.40 wt % of the cosmetic formula, about 0.15 wt % to about 0.30 wt %, less than, equal to, or greater than about 0.05 wt %, 0.10, 0.15, 0.20, 0.25, 0.30, 0.35, or about 0.40 wt %. Within the cosmetic formula, xanthan gum is in a range of from about 0.05 wt % to about 0.30 wt % of the cosmetic formula, 0.10 wt % to about 0.20 wt %, less than, equal to, or greater than about 0.05 wt %, 0.10, 0.15, 0.20, 0.25, or about 0.30 wt %. Within the cosmetic formula, the mixture of hydroxyethyl acrylate/sodium acryloyldimethyl taurate copolymer & sorbitan isostearate is in a range of from about 0.15 wt % to about 0.50 wt % of the cosmetic formula, about 0.20 wt % to about 0.40 wt %, less than, equal to, or greater than about 0.15 wt %, 0.20, 0.25, 0.30, 0.35, 0.40, 0.45, or about 0.50 wt %.

The cosmetic formulation can include any number of suitable surfactants. The surfactant can help to tune the feel, texture, and spreadability of the cosmetic formulation. An example of a suitable surfactant can include sodium stearoyl glutamate. The surfactant can be in a range of from about 0.1 wt % to about 2 wt % of the cosmetic formulation, about 0.1 wt % to about 1.5 wt %, less than, equal to, or greater than about 0.1 wt %, 0.2, 0.3, 0,4, 0.5, 0.6, 0.7, 0.8, 0.9, 1, 1.1, 1.2, 1.3, 1.4, 1.5, 1.6, 1.7, 1.8, 1.9, or about 2 wt %.

The surfactant can be a single surfactant or can be a mixture of many other suitable surfactants in addition to sodium stearoyl glutamate. For example the surfactant can include nonionic surfactants.

Suitable nonionic surfactants are disclosed in U.S. Pat. No. 3,929,678, Laughlin et al., issued Dec. 30, 1975, and U.S. Pat. No. 4,285,841, Barrat et al, issued Aug. 25, 1981. Exemplary, non-limiting classes of useful nonionic surfactants include Cs-Ci alkyl ethoxylates, with about 1-22 ethylene oxide units, including the so-called narrow peaked alkyl ethoxylates and C₆-C₁₂ alkyl phenol alkoxylates, particularly ethoxylates and mixed ethoxylates/propoxylates, alkyl dialkyl amine oxides, alkanoyl glucose amides, and mixtures thereof. Other useful nonionic surfactants are polyethylene, polypropylene, and polybutylene oxide condensates of alkyl phenols. Commercially available nonionic surfactants of this type include Igepal® CO-630, marketed by the GAF Corporation; and Triton® X45, X-114, X-100, and X-102, all marketed by the Rohm & Haas Company. These compounds are commonly referred to as alkyl phenol alkoxylates, preferably alkyl phenol ethoxylates. Further useful nonionic surfactants are the condensation products of aliphatic alcohols with from about 1 to about 25 moles of ethylene oxide. The alkyl chain of the aliphatic alcohol can either be straight or branched, primary or secondary, and generally contains from about 8 to about 22 carbon atoms. Examples of commercially available nonionic surfactants of this type include Tergitol® 15-S-9 (the condensation product of C₁₁-C₁₅ linear secondary alcohol with 9 moles ethylene oxide), Tergitol® 24-L-6 NMW (the condensation product of C₁₂-C₁₄ primary alcohol with 6 moles ethylene oxide with a narrow molecular weight distribution), both marketed by The Dow Chemical Corporation; Neodol® 45-9 (the condensation product of C₁₄-C₁₅ linear alcohol with 9 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, and Kyro® EOB (the condensation product of C₁₃-C₁₅ alcohol with 9 moles ethylene oxide), marketed by The Procter & Gamble Company. Other commercially available nonionic surfactants include Dobanol 91-8® marketed by Shell Chemical Co. and Genapol UD-080® marketed by Hoechst. This category of nonionic surfactant is referred to generally as “alkyl ethoxylates.” Other useful nonionic surfactants are the condensation products of ethylene oxide with a hydrophobic base formed by the condensation of propylene oxide with propylene glycol. Examples of compounds of this type include certain of the commercially-available Pluronic® surfactants, marketed by BASF. Further useful surfactants are the condensation products of ethylene oxide with the product resulting from the reaction of propylene oxide and ethylenediamine. Examples of this type of nonionic surfactants include certain of the commercially available Tetronic® compounds, marketed by BASF. Semi-polar nonionic surfactants are a special category of nonionic surfactants which include water-soluble amine oxides. These amine oxide surfactants in particular include C₁₀-C₁₈ alkyl dimethyl amine oxides and C₈-C₁₂ alkoxy ethyl dihydroxy ethyl amine oxides. Other nonionic surfactants are alkylpolysaccharides. Any reducing saccharide containing 5 or 6 carbon atoms can be used, e.g., glucose, galactose and galactosyl moieties can be substituted for the glucosyl moieties. Fatty acid amide surfactants, C₁₂-C₁₈ betaines and sulfobetaines (sultaines) are also knows surfactants.

Further useful surfactants are amine oxide surfactants. Commercially available amine oxides are the solid, dihydrate ADMOX 16 and ADMOX 18, ADMOX 12 and especially ADMOX 14 from Ethyl Corp. Other surfactants include dodecyidimethylamine oxide dihydrate, hexadecyidimethylamine oxide dihydrate, octadecyidimethylamine oxide dihydrate, hexadecyltris(ethyleneoxy)dimethyl-amine oxide, tetradecyidimethylamine oxide dihydrate, and mixtures thereof.

Other useful surfactants are biodegradably branched surfactants are more fully disclosed in WO98/23712 A published Jun. 4, 1998; WO97/38957 A published Oct. 23, 1997; WO97/38956 A published Oct. 23, 1997; WO97/39091 A published Oct. 23, 1997; WO97/39089 A published Oct. 23, 1997; WO97/39088 A published Oct. 23, 1997; WO97/39087 A1 published Oct. 23, 1997; WO97/38972 A published Oct. 23, 1997; WO 98/23566 A Shell, published Jun. 4, 1998.

Specifically useful surfactants can include a polyglyceryl fatty acid ester, polyglyceryl-3 diisostearate, polyglyceryl-4 isostearate, polyglyceryl-3 cocoate, polyglyceryl-6 Ricinoleate, polyglyceryl-3 stearate polyglyceryl-10 stearate, polyglyceryl-10 oleate, polyglyceryl-4 diisostearate/polyhydroxystearate/sebacate, polyglyceryl-2 dipolyhydroxystearate, polyglyceryl-3 poliricinoleate, polyglyceryl-6 stearate (and) polyglyceryl 6-behenate, sorbitan esters, sucrose esters, alkyl polyglycosides, fatty acid glucamides, or a mixture thereof. If the surfactant includes a polyglyceryl fatty acid ester, the polyglyceryl fatty acid ester can include a mixture of polyglyceryl-4 laurate/sebacate; polyglyceryl-6 caprylate/caprate; and water. The polyglyceryl fatty acid ester can alternatively or additionally include a mixture of polyglyceryl-4 laurate/sebacate, polyglyceryl-4 caprylate/caprate; and water. The polyglyceryl fatty acid ester can alternatively or additionally include polyglyceryl-4 caprate. Other ester surfactants can include a mixture of caprylic and capryc triglyceride, PEG-7 glyceryl cocoate, isosteryl neopentanoate, ethylhexyl palmitate, diethylhexyl adipate, diethylhexyl succinate, isotearyl isostearate, or a mixture thereof.

In some examples, the surfactant can be classified as an emulsifier. The emulsifier can be in a range of from about 1 wt % to about 5 wt % of the formula, about 2 wt % to about 3.5 wt % of the composition, less than, equal to, or greater than about 1 wt %, 1.5, 2, 2.5, 3, 3.5, 4, 4.5, or about 5 wt % of the composition. Emulsifiers typically employed in the compositions of the present invention include anionic, nonionic and cationic emulsifiers. See, e.g., Encyclopedia of Chemical Technology, KIRK-OTHMER, volume 22, pp. 333-432, 3rd edition, 1979, Wiley, for the definition of the properties and (emulsifying) functions of the emulsifiers, in particular pp. 347-377 of this publication regarding anionic and nonionic emulsifiers. Examples of emulsifiers useful in the formula of the invention include as nonionic emulsifiers, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic emulsifiers, C₁₆-C₃₀ fatty acids neutralized by amines, ammonia or the alkali metal salts thereof. Examples of cationic emulsifiers include quaternary amines, amine oxides and amines, e.g., alkyl amines, alkyl imidazolines, ethoxylated amines, quaternary compounds, and quaternized esters. Cationic emulsifiers may also provide a conditioning effect. A particularly useful blend of emulsifiers is a blend of ABIL EM 90 and sorbitan olivate. Additional examples of emulsifiers include sodium stearyl glutamate; a mixture of C12-16 alcohols, palmitic acid, and hydrogenated lecithin; hydrogenated coco-glycerides; a mixture of polyglyceryl-6 stearate and polyglyceryl-6 behenate; glyceryl oleate citrate; a mixture of arachidyl alcohol, behenyl alcohol, and arachidyl glucoside; a mixture of ethylhexyl olivate, sodium acrylates copolymer, and polyglyceryl-4 olivate; a mixture of PEG-100 and glyceryl stearate; glyceryl stearate; PEG-2 stearate; or a mixture thereof.

The oil phase is in a range of from about 2 wt % to about 15 wt % of the cosmetic formula, about 2 wt % to about 10 wt %, less than, equal to, or greater than about 2 wt %, 3, 4, 5, 6, 7, 8, 9, 10, 11, 12, 13, 14, or about 15 wt %. In a particular example, the oil phase can include caprylic capric triglyceride, a C15-19 alkane. Within the cosmetic formulation caprylic capric triglyceride is in a range of from about 1 wt % to about 5 wt % of the cosmetic formula, about 2 wt % to about 4 wt %, less than, equal to, or greater than about 1 wt %, 1.5, 2, 2.5, 3, 3.5, 4, 4.5, or about 5 wt %. Within the cosmetic formulation, the C15-19 alkane is in a range of from about 1 wt % to about 5 wt % of the cosmetic formula, about 2 wt % to about 4 wt %, less than, equal to, or greater than about 1 wt %, 1.5, 2, 2.5, 3, 3.5, 4, 4.5, or about 5 wt %. The oil phase can additionally, or alternatively, include a mixture of olus, vegetable oil, hydrogenated vegetable oil, candelilla wax; arggania spinosa kernel oil; a mixture of helianthus annuus seed oil, tocopherol, and vegetable oil; cocos necifera oil; crambe abyssinica seed oil; or a mixture thereof.

The active component is in a range of from about 15 wt % to about 35 wt % of the cosmetic formula, about 20 wt % to about 30 wt %, less than, equal to, or greater than 15 wt %, 16, 171, 18, 19, 20, 21, 22, 23, 24, 25, 26, 27, 28, 29, 30, 31, 32, 33, 34, or about 35 wt %. The active component can include any component or mixture of components to achieve a desired result. Examples of active components include lactic acid, a hyaluronic acid, vitamin E, saccharomyces/xylinum/black tea ferment, diglucosyl gallic acid, adenosine, panthenol, dimethylmethoxy chromanol, alpha-glucan oligosaccharide, Thermus thermophilus ferment, hydrolyzed hyaluronic acid, caffeine, acetyl di-peptide-1 cetyl ester, glycerin, niacinamide, swertia chirata extract, creatine, or a mixture thereof. The mixture of gels described and surfactant(s) herein above, allow for the relatively high amount of active component. The ability of the formulation to have a high amount of active component can enhance the formulation's effectiveness.

In some examples, the formulation can include a chelant or a mixture of two or more chelants. Examples of suitable chelants can include tetrasodium glutamate diacetate (GLDA), ethylenediaminetetraacetic acid (EDTA), ethylenediamine-N, N′-disuccinic acid (EDDS), NTA, EDDS, IDS, methylglycinediacetic acid (MGDA), L-glutamic acid N,N-diacetic acid (GLDA), ethylenediamine-N,N-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), methylglycine N,N-diacetic acid trisodium salt (MGDA), sodium gluconate, or a mixture thereof.

The formulas herein can contain a variety of other optional components suitable for rendering such compositions more cosmetically or aesthetically acceptable or to provide them with additional usage benefits. Such conventional optional ingredients are well-known to those skilled in the art. These include any cosmetically acceptable ingredients such as those found in the CTFA International Cosmetic Ingredient Dictionary and Handbook, 7th edition, edited by Wenninger and McEwen, (The Cosmetic, Toiletry, and Fragrance Association, Inc., Washington, D.C., 1997). As used herein “cosmetically acceptable” means a material (e.g., compound or composition) which is suitable for use in contact with skin, hair or other suitable substrate.

A pH of the cosmetic formula is in a range of from about 5 to about 7, about 5.5 to about 6.5, less than, equal to, or greater than about 5, 5.5, 6, 6.5, or about 7.

The cosmetic formula can be made using any suitable method. For example, the cosmetic formula can be made by combining the gel mixture, surfactant, and oil phase. All the components can be stirred or otherwise mixed to form the final cosmetic formula. The cosmetic formula can be used in a variety of ways. For example, the cosmetic formula can be topically applied to a user's skin. The cosmetic formula can be topically applied by hand, with an applicator, or both. Examples of applicators can include brushes, pads, sponges, or the like. The cosmetic formula can be applied once daily (e.g., once every 24 hours) or multiple times throughout the day. For example, the cosmetic formula can be applied two times, three times, four times, and so on within a 24 hour period.

### EXAMPLES

Various embodiments of the present invention can be better understood by reference to the following Examples which are offered by way of illustration. The present invention is not limited to the Examples given herein.

Various cosmetic formulas according to the instant disclosure will be tested according to sensory testing protocol before (“pick-up”), during, and after application of the cosmetic formulation. The sensory testing protocol can include testing for appearance, texture, and odor at various timings.

For the pick-up testing, e.g., before application, the cosmetic formulation will be tested in appearance for glossiness, and in texture for firmness, stickiness, and stringiness.

For testing at pick-up, about 100 μl of the sample formulation is dropped off the thumb of each panelist conducting the sensory testing. This process is repeated for testing of glossiness, firmness, stickiness, and stringiness of the pre-application “pick-up” testing.

Pick-up Glossiness. The cosmetic formulation glossiness will be tested to see the quantity of light reflected by the product. The glossiness of the formulation will be tested at pick-up. In the test for glossiness, the gloss of the product is assessed by looking at the product under white light, and specifically analyzed while the 100 μl of the sample formulation is on the thumb of the panelist. The product is assigned a value on a scale of mat to glossy.

Pick-up Firmness. The cosmetic formulation firmness will be tested to see the strength required to compress the product. Here, the 100 μl of the sample formulation on the thumb will be compressed softly between the thumb and forefinger. The panelist will assign the sample formulation a score of low to high.

Pick-up Stickiness. The cosmetic formulation firmness will be tested to see the strength required to remove the forefinger from the thumb. Here, following the test for firmness, the 100 μl of the sample formulation will be on the compressed thumb and forefinger, which will be softly separated. The panelist will assign the sample formulation a score of low to high.

Pick-up Stringiness. The cosmetic formulation firmness will be tested to see the quantity of the product being stretched, rather than broken, when separating the fingers. Here, the 100 μl of the sample formulation will be compressed between the thumb and the forefinger and separated once. The distance between the fingers will be equivalent to one finger. The panelist will assign the sample formulation a score of low to high.

It is expected that a majority of participants evaluating the serum will find that the serum feels comfortable when applied to the skin. It is further expected that the measured spreadability of the serum will allow the serum to not run to an undesirable degree following application.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Aspects

The following exemplary aspects are provided, the numbering of which is not to be construed as designating levels of importance:
Aspect 1 provides a cosmetic formula comprising:
   a gel mixture in a range of from about 0.1 wt % to about 2 wt % of the cosmetic formula and comprising:
      sphingomas ferment extract;
      xanthan gum; and
      a mixture of hydroxyethyl acrylate/sodium acryloyldimethyl taurate copolymer & sorbitan isostearate;
   a surfactant in a range of from about 0.1 wt % to about 2 wt % of the cosmetic formula and comprising sodium stearoyl glutamate;
   an oil phase in a range of from about 2 wt % to about 15 wt % of the cosmetic formula and comprising:
      caprylic capric triglyceride;
      a C15-19 alkane; and
         an active component, wherein
   a pH of the cosmetic formula is in a range of from about 5 to about 7;
   a viscosity of the cosmetic formula is in a range of from about 1500 cPs to about 5000 cPs; and
   the cosmetic formula is sufficiently stable for at least 2 months at a temperature of about 45 °C.
Aspect 2 provides the cosmetic formula of Aspect 1, wherein the gel mixture is in a range of from about 0.3 wt % to about 1 wt % of the cosmetic formula.
Aspect 3 provides the cosmetic formula of any of Aspects 1 or 2, wherein
   sphingomas ferment extract is in a range of from about 0.05 wt % to about 0.40 wt % of the cosmetic formula;
   xanthan gum is in a range of from about 0.05 wt % to about 0.30 wt % of the cosmetic formula; and
   the mixture of hydroxyethyl acrylate/sodium acryloyldimethyl taurate copolymer & sorbitan isostearate is in a range of from about 0.15 wt % to about 0.50 wt % of the cosmetic formula.
Aspect 4 provides the cosmetic formula of any of Aspects 1-3, wherein
   sphingomas ferment extract is in a range of from about 0.15 wt % to about 0.30 wt % of the cosmetic formula;
   xanthan gum is in a range of from about 0.10 wt % to about 0.20 wt % of the cosmetic formula; and
   the mixture of hydroxyethyl acrylate/sodium acryloyldimethyl taurate copolymer & sorbitan isostearate is in a range of from about 0.20 wt % to about 0.40 wt % of the cosmetic formula.
Aspect 5 provides the cosmetic formula of any of Aspects 1-4, wherein the surfactant component is in a range of from about 0.1 wt % to about 1.5 wt % of the cosmetic formula.
Aspect 6 provides the cosmetic formula of any of Aspects 1-5, wherein the oil phase is in a range of from about 2 wt % to about 10 wt % of the cosmetic formula.
Aspect 7 provides the cosmetic formula of any of Aspects 1-6, wherein the caprylic capric triglyceride is in a range of from about 1 wt % to about 5 wt % of the cosmetic formula.
Aspect 8 provides the cosmetic formula of any of Aspects 1-7, wherein the C15-19 alkane is in a range of from about 1 wt % to about 5 wt % of the cosmetic formula.
Aspect 9 provides the cosmetic formula of any of Aspects 1-8, wherein the active component is in a range of from about 15 wt % to about 35 wt % of the cosmetic formula.
Aspect 10 provides the cosmetic formula of any of Aspects 1-9, wherein the active component is in a range of from about 20 wt % to about 30 wt % of the cosmetic formula.
Aspect 11 provides the cosmetic formula of any of Aspects 1-10, wherein the active component comprises lactic acid, a hydrolyzed hyaluronic acid, vitamin E, saccharomyces/xylinum/black tea ferment, diglucosyl gallic acid, or a mixture thereof.
Aspect 12 provides the cosmetic formula of any of Aspects 1-11, wherein the cosmetic formula is a serum.
Aspect 13 provides the cosmetic formula of any of Aspects 1-12, wherein the cosmetic formula can be used for skin fine lines reduction, improving skin firmness, evening skin tone, strengthening skin barrier, hydrating skin, enhancing skin natural radiance, or a combination thereof.
Aspect 14 provides a method of making the cosmetic formula of any of Aspects 1-13, the method comprising
   combining the gel mixture, surfactant, and oil phase.
Aspect 15 provides a method of using the cosmetic formula of any of Aspects 1-14, the method comprising applying the cosmetic formula to a user's skin.
Aspect 16 provides the method of Aspect 15, wherein the cosmetic formula is applied twice within 24 hours.

1. A O/W serum comprising:
a gel mixture in a range of from about 0.1 wt % to about 2 wt % of the cosmetic formula and comprising:
Sphingomonas ferment extract;
xanthan gum; and
a mixture of hydroxyethyl acrylate/sodium acryloyldimethyl taurate copolymer & sorbitan isostearate;
a surfactant in a range of from about 0.1 wt % to about 2 wt % of the cosmetic formula and comprising sodium stearoyl glutamate;
an oil phase in a range of from about 2 wt % to about 15 wt % of the cosmetic formula and comprising:
caprylic/capric triglyceride;
a C15-19 alkane; and
from 15% to 35% of one or more active components, wherein
a pH of the cosmetic formula is in a range of from about 5 to about 7;
a viscosity of the cosmetic formula is in a range of from about 1500 cPs to about 5000 cPs; and
the cosmetic formula is stable for at least 2 months at a temperature of about 45 C.

2. The cosmetic formula of claim 1, wherein the gel mixture is in a range of from about 0.3 wt % to about 1 wt % of the cosmetic formula.

3. The cosmetic formula of claim 1, wherein
Sphingomonas ferment extract is in a range of from about 0.05 wt % to about 0.40 wt % of the cosmetic formula;
xanthan gum is in a range of from about 0.05 wt % to about 0.30 wt % of the cosmetic formula; and
the mixture of hydroxyethyl acrylate/sodium acryloyldimethyl taurate copolymer & sorbitan isostearate is in a range of from about 0.15 wt % to about 0.50 wt % of the cosmetic formula.

4. The cosmetic formula of claim 1, wherein
Sphingomonas ferment extract is in a range of from about 0.15 wt % to about 0.30 wt % of the cosmetic formula;
xanthan gum is in a range of from about 0.10 wt % to about 0.20 wt % of the cosmetic formula; and
the mixture of hydroxyethyl acrylate/sodium acryloyldimethyl taurate copolymer & sorbitan isostearate is in a range of from about 0.20 wt % to about 0.40 wt % of the cosmetic formula.

5. The cosmetic formula of any of claim 1, wherein the surfactant component is in a range of from about 0.1 wt % to about 1.5 wt % of the cosmetic formula.

6. The cosmetic formula of claim 1, wherein the oil phase is in a range of from about 2 wt % to about 10 wt % of the cosmetic formula.

7. The cosmetic formula of claim 1, wherein the caprylic capric triglyceride is in a range of from about 1 wt % to about 5 wt % of the cosmetic formula.

8. The cosmetic formula of claim 1, wherein the C15-19 alkane is in a range of from about 1 wt % to about 5 wt % of the cosmetic formula.

9. The cosmetic formula of claim 1, wherein the active component is in a range of from about 15 wt % to about 35 wt % of the cosmetic formula.

10. The cosmetic formula of claim 1, wherein the active component is in a range of from about 20 wt % to about 30 wt % of the cosmetic formula.

11. The cosmetic formula of claim 1, wherein the active component comprises lactic acid, a hyaluronic acid, vitamin E, saccharomyces/xylinum/black tea ferment, diglucosyl gallic acid, or a mixture thereof.

12. The cosmetic formula of claim 1, wherein the cosmetic formula can be used for skin fine lines reduction, improving skin firmness, evening skin tone, strengthening skin barrier, hydrating skin, enhancing skin natural radiance, or a combination thereof.

13. A method of making the cosmetic formula of claim 1, the method comprising
combining the gel mixture, surfactant, and oil phase.

14. A method of using the cosmetic formula of claim 1, the method comprising applying the cosmetic formula to a user's skin.

15. The method of claim 14, wherein the cosmetic formula is applied twice within 24 hours.
